(19) United States
(12) Patent Application Publication
(10) Pub. No.: US 2022/0387206 A1
(43) Pub. Date: Dec. 8, 2022

(54) A POLYSILOXANES ORTHOPAEDIC IMMOBILIZER

(71) Applicant: Pankajkumar Kumanbhai CHHATRALA, Surendranagar (IN)

(72) Inventors: Pankajkumar Kumanbhai CHHATRALA, Surendranagar (IN); Manisha PAWAR, Surendranagar (IN); Chintan SAPOVADIYA, Surendranagar (IN); Heena PRAJAPATI, Surendranagar (IN); Sachin MAKWANA, Surendranagar (IN)

(21) Appl. No.: 17/793,425

(22) PCT Filed: Jan. 20, 2021
(86) PCT No.: PCT/IB2021/050406

§ 371 Date: Jul. 18, 2022

(30) Foreign Application Priority Data
Jan. 17, 2020 (IN) 202021002189

(51) Int. Cl. A61F 5/05; A61F 5/01; C07F 7/08

(52) U.S. Cl. CPC A61F 5/05; A61F 5/0104; C07F 7/0838

(57) ABSTRACT

A polysiloxanes orthopaedic immobilizer relates to a construction and method for forming an orthopaedic apparatus and more particularly, to medical shell for preventing and supporting fractured body part. Moreover, the present invention is to provide a curable orthopaedic apparatus for immobilizing fractured body parts wherein said apparatus converts in rigid immobilizer by applying visible light. An apparatus comprising polygon hollow tube in which composition of resin is filled for curing. Moreover, an apparatus has a maximum light distribution tendency, viscus fluid distribution tendency and comprises a safe layer prevents direct contact of skin to said apparatus. [FIG. 1]

### FIELD OF THE INVENTION

The present invention relates to a construction and method for forming an orthopaedic apparatus and more particularly, to medical shell for preventing and supporting fractured body part.

### BACKGROUND OF THE INVENTION

A bone fracture is a medical condition in which there is a partial or complete break in the continuity of the bone. In more severe cases, the bone may be broken into several pieces. A bone fracture may be the result of high force impact or stress, or a minimal trauma injury as a result of certain medical conditions that weaken the bones, such as osteoporosis, osteopenia, bone cancer, or orthogenesis imperfecta, in such cases Fracture is termed a pathologic fracture.

The management of fractures and injuries to the extremities has a long and colourful history in medicine. Prior to the invention of plaster of Paris by the Flemish army surgeon Mathieson in 1852, the stabilization of broken bones and sundry joint injuries was a haphazard affair using pieces of wood, branches, and any rigid material that might be available. Plaster of Paris was rubbed into muslin or linen cloth prior to its use, “a tedious process”, and wrapped around the injured limb to provide stability. When the anhydrous calcium sulphate was recombined with water, the reaction produced the slow drying, but stiff gypsum. Manufactured rolls of plaster of Paris cast material were not available until the mid-1900's. The casts made in this way were messy to apply, often extremely bulky, could be very difficult to remove, would break down with walking, fell apart in water, were heavy, and made visualization of a fracture difficult with x-ray. Yet at the time they were a major step forward in the medical management of these unstable musculoskeletal injuries.

Surgery in the first half of the nineteenth century was primarily dominated by pain and fear of lethal infection. Therefore, the absolute majority of fractures and dislocations were treated non-operatively. Development of operative treatment of fractures was influenced by three major prior art said: anaesthesia (1846), antisepsis (1865) and X-rays (1895). The first to use external fixation is traditionally considered to be Malgaigne (1843).

There are too many types of treatment of bone fracture which describe below but every prior art has disadvantages and not so efficient for treatment of bone fracture and dislocation of bone.

Over a Period of Time,

1. Plaster of Paris

Plaster casts were used consistently and devotedly until the 1980's when fiberglass materials became available. Though more expensive and somewhat more difficult to work with, fiberglass soon became the preferred casting material in many clinics and hospitals and remains so today. It offered increased lightness, somewhat better visibility under x-ray, and was resistant to softening if wet. Unlike plaster rolls or strips which could remain open on the shelf, fiberglass would gradually harden if exposed to air and needed to be packaged in airtight bags.

The US patent number U.S. Pat. No. 3,332,416A discloses the method of making elastic plaster of paris bandages comprising taking a loose weaver fabric having both elastic and inelastic warp threads, in which said elastic warp threads are substantially shorter when relaxed then said inelastic warp threads, extending said fabric under tension to substantially the full extent permitted by said inelastic warp threads, and while so extended coating said fabric with a liquid slurry of Plaster of Paris and then drying said coated fabric without relaxing the same, to hold said fabric in said extended state after said tension is removed and until again wetted.

In medicine, cast of POP still uses, but POP has more disadvantages as below:
POP cast has weight and bulkier makes patient's life more difficult.
POP cast is not water resistance as patient has to take more care.
POP cast is not breathable as patient suffers by itchiness.
Exercise any joints that aren't covered by the cast—such as elbow, knee, fingers or toes—to help improve your circulation.
POP cast requires soft surface against bony prominence in order to prevent cast sore.
The process for POP cast wearing is very time consuming and requires more time to harden.

2. Glass Fiber or Synthetic Cast

A fiberglass cast is a plaster cast made from fiberglass material which is lighter than traditional POP cast, made up from synthetic glassfibers impregnated with water activated resin alternative plaster of Paris. Fiberglass is also called glass-reinforced plastic [GRP] or glass fiber reinforced plastic [GFRP] is a fiber reinforced polymer made of a plastic matrix reinforced by fine fibers of glass. It comes in rolls similar to fiberglass and is wrapped around the injured extremity over a cotton or fabric padding under-layer in the same fashion as plaster and fiberglass. It too is water activated. It is slightly more expensive than fiberglass, but has advantages of comfort and light weight compared to Plaster of Paris Cast For the purposes of this discussion it is important to define immobilizers, casts, and braces. While these are all used to Support body parts, they are separate and distinct products, each constructed differently and for disparate uses. U.S. Pat. No. 4,323,061 discloses a cast substrate made from a combination of glass fibres and a second fibre such as cotton, flax, rayon, wool, acrylic resin, nylon, Teflon or polyester. The purpose of the second fibre in the substrate is to hold the curable resin on the substrate.

The fiberglass cast however is not applied in acute settings this cast is less accommodating to swelling or where the reduction of the fragments necessitates moulding. Moreover, the fiberglass cast is less yielding to moulding and to hold fracture fragments, molding is required.

A variation on fiberglass is the Delta-cast brand (distributed by BSN Medical) of roll fiberglass casting tape material that is more pliable and less rigid than fiberglass, also known as Soft cast, has improved radiolucency and can be cut with a scissors as well as with a cast saw. It comes in rolls similar to fiberglass and is wrapped around the injured extremity over a cotton or fabric padding under-layer in the same fashion as plaster and fiberglass. It is also water activated same like rigid Fiberglass cast. It is slightly more expensive than fiberglass, but has advantages of comfort and enough flexibility to be removed in some instances.

All of these casting materials disclosed in prior-art having fiberglass with different reactive polymers suffer from certain disadvantages. One of the major disadvantages is the conformability of the casting tape to the body of the patient. Conformability is the characteristic of the casting tape which has been defined as that property which describes the ability of the bandage or casting tape to adapt to or intimately lay down against the compound curves and protrusions of a body member. This leads to the requirement of applying plaster of Paris as a primary casting material and fiberglass as a secondary casting tape. Which makes the healing process time-intensive, labour-intensive and expensive as it involves cutting, aligning in a skilled way. Few inventors have disclosed new device or design of liner and glass fibres cast to make it washable cast. i.e U.S. Pat. No. 6,585,671B2. It discloses a sleeve that can be water activated and rolled around fractured limb. Major disadvantage is that it requires a stockinette fabric in order to protect skin from resin contact. As inventor claims it to be washable, dead skin accumulation in stockinette is unavoidable and as a result maceration of dead skin cells foul smelling comes from cast.

3. Thermoplastic Immobilization

The common thermoplastic materials, polyethylene, PVC and polystyrene are therefore now also available in the developing countries. During the 1960's thermoplastic materials such as polyethylene and polypropylene became more commonly used in prosthetics and orthotics. The drop foot brace was the first important application of thermoplastics in orthotics and is perhaps still the most produced orthopaedic orthosis made of thermoplastic material. In addition, thermoplastics are commonly used with metal joints to produce long-leg braces (knee-ankle-foot orthoses) and for the manufacture of spinal orthoses (Oberg, 1988). During the 1970's, when new innovations in prosthetic sockets occurred more and more, thermoplastic materials were used instead of fibre-reinforced resins. Because of the properties of thermoplastic materials, the concept of flexible sockets could be introduced. Another advantage of introducing thermoplastic materials in prosthetics was the possibility of making much lighter prostheses.

The primary disadvantage of using thermoplastics instead of materials such as metal is their relatively low melting point. Certain types of low-quality thermoplastics can melt when they're exposed to the sun for extended periods or hot climate area in summer Furthermore, thermoplastics can have poor resistance to organic solvents, hydrocarbons, and highly polar solvents.

Thermoplastics are susceptible to creep, which occurs when the material stretches and weakens under exposure to long-term stress loads. The susceptibility to creep is further exacerbated by the lower melting temperature of the material. Other types of thermoplastics, such as composites, can fracture instead of becoming deformed under high-stress conditions.

4. Resin Injection/Two-Part Resin Immobilization

US Patent Application No: U.S. Pat. No. 4,483,332; Bruce Rind disclosed a method of making an orthopaedic cast, in which interconnected double lumen tubes were used with at least one port through with resinous or epoxy material injected and which is being hardened in 5-10 minutes. Out of double lumen, one lumen is filled with resin while other lumen can be adjusted with Air filling with pressure. Inner layer provides cushion, insulation against heat generation during hardening of material. However, it is difficult to maintain air inside said lumen because in real life, there is a chance of getting punctures and pressure will be released, which makes loose cast and ineffective for intended use of immobilization. Another major issue with the above invention is that Tubes are made up of plastic material i.e. polyethylene or polypropylene which makes it transparent to see through tubes, but on the other side, even being flexible, it is difficult to make shape of extremity contours. Which makes poor fitting of Cast over extremity and as a result it becomes ineffective or tiresome process. In addition to that, inventor claimed that interconnected tube should be devoid of air. In practical life, it is very difficult and costly to maintain such condition of tube hence it makes economically non-viable.

Another similar invention is US20190105423, Moy et al. disclosed similar invention with improved method of injection. Inventor has disclosed pouch of harden able material which can be mixed and injected into interconnected tubes which are wrapped over a broken hand. As per claims, this invention gives choices for variety of customization for pigments. And apart from that, to overcome issue of air leakage in second lumen mentioned in US 448332, Moy et all used cushion foam to insulate and cushion inner side of cast. However same like US448332, US20190105423 also has tiresome process and difficult to make conform shape of extremity.

To overcome fitting issues identified in US 448332 and US20190105423, US 2017/0079830 A1, Chhatrala et al, disclosed and new invention in which inventor used elastomer material shell for better conforming of the shape of extremity. They claimed that being elastic material, said cast can be fitted and conformed to the shape of extremity when it is stretched and applied over extremity. In above invention, Material is being received from one port same like U.S. Pat. No. 4,483,332 and US20190105423. Chhatrala et al. claimed that because if elastic material for tube construction, it is easier to conform the shape of the extremity.

While considering inventions disclosed in U.S. Pat. No. 4,483,332, US20190105423 and US 2017/0079830 A1, common problem is, time consuming process of making cast or orthopaedic immobilization at point of care. In current scenario, Healthcare professional's time is very precious and saving even 4-5 minutes makes a huge difference in quality and reach of healthcare/medical care delivery in community. Not only that, as above inventions require additional skills to follow standard process in order to achieve desirable result, in busy schedule one cannot neglect the chance of process error in above invention. Additionally, none of above commented on weight bearing of the cast as typically for lower extremity cast/brace weight bearing is required and it required rigid and strong material which can withstand weight of person taking treatment. Breakage of immobilizer/cast reduces effectiveness of treatment outcome.

5. Photo Curable Immobilization

Use of photo curable resin in orthopaedic immobilization was initially disclosed by Carl in U.S. Pat. No. 4,512,340, as disclosed in invention, Fiberglass fabric is impregnated with light curable resin and applied like a bandage and then exposed with incandescent light in order to cure and harden cast. Disclosed Process is tiresome and lengthy process, in practical life it never got popularity because of added step of curing with incandescent lamp. With that comparison, water curable resin was much more convenient and it also reduces additional step and equipment. Another drawback was that above invention was about replacing resin component however problem with respect to wash-ability and breathability is unsolved through this invention.

Just after the above invention, Glass fiber tape with water activated polyol resin got popular in the market because of its stability, fast curing and rapid gain of strength after curing.

Apart from the use of photo curable resin in casting tape, another prior art disclosed in by Jensen, Torben in EP2323702B1 in which inventor disclosed method of making an orthopaedic cast from photo curable resin. In this invention the same like U.S. Pat. No. 4,512,340, method of making cast which involves several steps i e making cast over limb, adjusting, forming and curing. All over it takes time for making whole cast. From disclosure in invention inventor have not specified wash ability and breathability of the cast made from mentioned invention.

6. 3D Printed Custom Cast/Immobilizer

2005/0015172A1 disclose a method through which custom cast is fabricated using systems and processes. Typically, this process makes accurate and custom immobilizer as specific to individual who needs treatment. Above invention has mentioned CT scan, X-ray or similar device to extract outer line of skin surface and after Computed Aided

Design, cast or Immobilizer is extracted in form of CAD design. There are several 3D printing technologies through which Immobilizer or Slab can be made on demand and said Immobilizer/slab is washable. Technology mentioned in this invention is ground breaking in terms of customization and skin hygiene. One of the biggest drawbacks of this invention is economic viability and other one is the time between diagnosis and treatment. This process requires expensive equipment i.e. 3D scanner, 3D printer, CAD software. To setting up this facility is cost intensive, in addition to that making one immobilizer or cast takes 12-36 hrs which is practically not scalable and viable in real life scenario.

7. Air Cast

Inventor in U.S. Pat. No. 8,226,585, disclosed a Brace support using inflatable bladder inside ridgid housing. Inventor claimed that disclosed invention is suitable as weight bearing cast and can be used as walking cast. Disclosed product is suitable for stable fractures and when patient is willing to mobilize with fracture. From disclosed invention, brace becomes bulky and heavy as there is a rigid housing outer side and inflatable layer inside, additionally disclosed brace cannot be washed when it is on the patient's leg. Because of that it restricts use of disclosed brace is not suitable for fractures where constant immobilization is required i.e. displaced fracture.

To solve the above problems the present invention providing a new immobilization system which is very quick ideally can be set up within 5-10 mins to save health care professional's time, without any messy procedure and it should be more hygienic, safe and comfortable for the patient as well. The present invention provides a cast which is prefilled by composite material, pre-fabricated as per different designs and size and applied directly to the fractured limb. Moreover, immobilizer of the present invention is timeless there is no need to pre-process like filling liquid or heating, or wrapping etc in order to make rigid cast. The present invention provides a immobilizer which has more strength than prior arts and customization is available. For cushioning and comfort closed cell synthetic rubber foam is used for immobilizer which prevents retention of water, fungal and bacterial growth and also provides insulation from exothermic reaction results at time of curing. One of the features is pre-fabricated immobilizer is stretchable due to its elasticity. Because of elasticity it can fit on to variable size difference.

### SUMMARY OF THE INVENTION

The principal object of the present invention is to provide a Quick orthopaedic Immobilization apparatus for immobilizing fractured body parts wherein said apparatus converts in rigid immobilizer support by applying visible light to said apparatus. Wherein Said apparatus should be easy and quick to use and Washable, Breathable and light weight after cured. An apparatus comprising polygon hollow tube in which composition of resin is filled for curing. Moreover, an apparatus has a maximum light distribution tendency, viscus fluid distribution tendency and comprises a layer that prevents direct contact of skin to said apparatus.

The second object of the present invention is to provide an apparatus wherein Top layer of immobilizer and Bottom layer of immobilizer configure a polygon hollow tube.

The third object of the present invention is to provide an immobilizer having polygon shape hollow tubes which provide more stretch ability, stable structure and deformation to the immobilizer.

One of the objects of the present invention is to provide an apparatus wherein polygon hollow tube comprises hollow cavity said cavity is inter connected in which composition of resin is filled at time of manufacturing.

One of the objects of the present invention is to provide an apparatus wherein polygon hollow tube provides minimum contact between skin and immobilizer. Further, immobilizer of the present invention provides at least 40% open skin surface compared to skin covered by immobilizer of prior art.

One of the objects of the present invention is to provide an apparatus made up from polysiloxanes having tendency of transmission of light rays. Moreover, said tendency of polysiloxanes allows visible light to reach at the base of the said apparatus. Hence photocuring of resin is possible in shortest time.

One of the objects of the present invention is to provide Closed cell synthetic foam beneath bottom layer. Closed cell Synthetic foam provided cushion to skin from hardened immobilizer. Closed cell synthetic Foam is non allercin and non-sensitizer as a result it gives comfort even after continuous contact with skin. Apart from that said foam does not absorb water or moisture hence it prevents fungal/bacterial vegetation and dead skin accumulation inside foam.

Another object of the present invention is to provide an apparatus wherein polygon shape of tube prevents tubes from collapsing and improve structural strength of said apparatus.

One of the objects of the present invention is to provide an apparatus wherein immobilizer structure arranges in polygon matrix form which provides structural strength after curing.

One of the objects of the present invention is to provide an apparatus wherein top layer of an immobilizer and bottom layer of an immobilizer is hydrophobic.

Another object of the present invention is to provide an apparatus wherein said apparatus comprises a Top Layer, Bottom layer, Foam layer, and protective layer which can be peeled of before use.

One of the objects of the present invention is to provide an apparatus wherein curing process starts after peeling protective layer provided on outer surface of said apparatus and on exposure of specific wavelength light or direct sunlight.

One of the objects of the present invention is to provide an apparatus wherein foam layer protects skin against heat generation at the time of polymerisation process of composition of resin.

Another object of the present invention is to provide an apparatus is made up of materials which permits X-ray hence suitable for X-Ray

One of the objects of the present invention is to provide an apparatus wherein different colours of dies is mixed with polysiloxanes and composition of resin for achieving aesthetic appearance.

One of the objects of the present invention is to provide an apparatus which comprises locking mechanism which fastens said apparatus at the time of wrapping around body parts.

One of the objects of the present invention is to provide an apparatus which is pre-fabricated and stretchable due to its elasticity and because of elasticity said immobilizer can fit on to variable size difference and various skin contours.

One of the objects of the present invention is to provide an apparatus having polygon matrix shape which is admitting air to the skin and allowing sweat to evaporate.

Another important object of the present invention is to provide an immobilizer which is washable and allows patient to maintain skin hygiene. Moreover, said immobilizer provides convenience for doing day to day work during treatment.

Another object of the present invention is a method for forming a matrix orthopaedic Immobilizer for immobilizing a body of a patient to allow healing.

### BRIEF DESCRIPTION OF THE DRAWINGS

The objects, features and advantages of the invention will best be understood from the following description of various embodiments thereof, selected for purposes of illustration, and shown in the accompanying figures.

FIG. 1. Illustrate isometric view of a polysiloxanes orthopaedic immobilizer.

FIG. 1A. discloses an exploded view of a polysiloxanes orthopaedic immobilizer.

FIG. 2. discloses cross-sectional view of said orthopaedic immobilizer.

FIG. 3. discloses different polygon shape of hollow tubing of orthopaedic immobilizer.

FIG. 4. discloses an orthopaedic immobilizer which is applied onto fractured ulna bone, radius bone of human body.

FIG. 5. discloses an orthopaedic immobilizer which is applied at the fracture elbow joint of human body.

FIG. 6. discloses an orthopaedic immobilizer which is applied at the fractured carpus.

FIG. 7. discloses an orthopaedic immobilizer which is applied at the fractured metacarpals and phalanges bones.

FIG. 8. discloses an orthopaedic immobilizer which is applied at the fractured femur, fibula and tibia bones.

FIG. 9. discloses an orthopaedic immobilizer which is applied at the cervical bone of the human.

FIG. 10. discloses an immobilizer having polygon shape hollow tubes.

FIG. 11. discloses a circular shape immobilizer.

FIG. 12. discloses square shape immobilizer.

FIG. 13. discloses triangular shape immobilizer.

### DETAILED DESCRIPTION OF THE INVENTION

The principal embodiment of the present invention is to provide a light curable orthopaedic apparatus for immobilizing fractured/injured body parts wherein said apparatus converts in rigid immobilizer by applying visible light to said apparatus. An apparatus comprising polygon hollow tube in which composition of resin is filled for curing. Moreover, an apparatus has a uniform light distribution tendency, a foam layer prevents direct contact of skin to said apparatus.

One of the embodiments of the present invention is to provide an apparatus wherein top layer of immobilizer and bottom layer of immobilizer forms a polygon hollow and interconnected tube wherein top layer and bottom layer is made up from polysiloxanes material. Moreover, top layer and bottom layer moulded with each other to create cavity said a polygon hollow tube. The shape of top layer and bottom layer is can be any matrix polygon said rectangle, pentagon, octagon, triangle, rhombus, circle, etc. but most preferable shape is hexagon matrix.

The hexagon shape defines as a shape or a plane having equal or unequal six straight sides and angles. The advantage of hexagon shape is matrix arrangement of said hexagon provides maximum strength and minimise use of material.

One of the embodiments of the present invention is to provide an apparatus wherein polygon hollow tube comprises cavity said cavity tubing in which composition of resin is filled wherein composition of resin will be cured by applying visible light onto it.

One of the embodiments of the present invention is to provide an apparatus wherein polygon hollow tube provides minimum contact between skin and immobilizer. At the time of curing process, heat is generated as chemical reaction accrues into composition of resin. The amount of heat generated having very high temperature which affects skin layer therefor polygon shape of said apparatus provides minimum contact or point contact between hollow tube and skin.

One of the embodiments of the present invention is to provide an apparatus made up from polysiloxanes having tendency of absorption and reflection. Moreover, said tendency of polysiloxanes allows visible light to reach at the base of the said apparatus. It is very important that for curing process, visible light has to reach at the bottom layer of said apparatus through liquid composition resin filled into polygon hollow tube. Due to polysiloxanes visible light can easily reflect and absorb within an apparatus which increases curing process. Moreover, polysiloxanes having tendency of minimum heat transfer rate so that heat generated at the time of curing does not affect to skin.

One of the embodiments of the present invention is to provide an apparatus wherein polygon hollow tube provides equal distribution of curable liquid or gel throughout the said apparatus without bubble formation. Polygon hollow tube provide less surface tension so that fluid flows easily into said tube.

Another embodiment of the present invention is to provide an apparatus wherein polygon shape of tube prevents tubes from collapsing and improve structural strength of said apparatus. Matrix form of said polygon provides structural strength as each edge and each corner of said polygon rigidly attached with another adjacent polygon and provide strong bond with each other.

One of the embodiments of the present invention is to provide an apparatus wherein rigid immobilizer structure arranges in polygon matrix form. Matrix form of said polygon provides structural strength as each edge and each corner of said polygon rigidly attached with another adjacent polygon and provide strong bond with each other.

One of the embodiments of the present invention is to provide an apparatus wherein top layer of an immobilizer and bottom layer of an immobilizer is hydrophobic.

One of the embodiments of the present invention is to provide an apparatus wherein curing process starts after peeling off protective layer provided on outer surface of said apparatus.

Another object of the present invention is to provide an apparatus having made of x-ray permeable materials so that x-ray radiograph can be taken without removing said apparatus from body surface. If cast of plaster of Paris required disengagement of cast to take x-ray picture. So that this is one of the advantages of the present invention which does not required disengagement of immobilizer.

One of the embodiments of the present invention is to provide an apparatus having polygon matrix shape which is admitting air to the skin and allowing sweat to evaporate.

One of the objects of the present invention is to provide an apparatus wherein different colours of pigments is mixed with polysiloxanes and composition of resin for achieving aesthetic appearance. This is one of the advantages of the present invention that user can get batter aesthetic view of immobilizer.

Another embodiment of the present invention is a method for forming a matrix orthopaedic immobilizer for immobilizing a body of a patient to allow healing with comfort.

One of the embodiments of the present invention is to provide an apparatus wherein Foam layer protects skin against heat generation at the time of polymerisation process of composition of resin.

One of the embodiments of the present invention is to provide an apparatus which is novel immobilization system which very quick ideally can be set up within 5-10 mins to save health care professional's time without any messy procedure.

One of the embodiments of the present invention is to provide an apparatus which is suitable to apply in emergency situation i.e. trauma room, road traffic accidents, war field, etc without any specific arrangements.

One of the embodiments of the present invention is to provide an apparatus which is more hygienic, safe and provide more comfort to the patient.

One of the embodiments of the present invention is to provide an apparatus wherein synthetic rubber foam is provided which prevents retention of water, fungal and bacterial growth and also provides insulation from exothermic reaction while curing.

One of the embodiments of the present invention is to provide an apparatus which is pre-fabricated and stretchable due to its elasticity and because of elasticity said immobilizer can fit on to variable size difference and different skin contours.

Another embodiment of the present invention is to provide an immobilizer which can be used without removable layer as well.

One of the embodiments of the present invention is to provide an immobilizer wherein composition of resin is cured between 750 nm to 380 nm of wavelength range. Most preferably wave length is 400 nm-470 nm. Moreover, the composition of resin might be cured with sunlight having range is between 100 nm to 1 mm of wave length.

Another important embodiment of the present invention is to provide an immobilizer having polygon shape hollow tubes which provide more stretch ability, stable structure and geometric advantages of polygon shape hollow tubes provides more deformation to the immobilizer. For an example, most preferably shape of hollow tubes of immobilizer is hexagon. If one can stretch a hexagon shape hollow tube then it configures a shape of rectangle which provides more stretch ability; whereas if one can stretch a rectangular shape of hollow tube then it will destroy the structure. So that, polygon shape hollow tube provides more stable structure

The polysiloxanes orthopaedic immobilizer of the present invention comprises padding layer made up of closed cell synthetic elastomer foam which gives insulation from exothermic heat produced during curing process as well as cushion to skin against hard shell formed after curing. Said closed cell foam is non-allergic and non-sensitize which suitable for long contact with skin. Moreover, It does not absorb water so that it allows user to wash immobilizer thoroughly without fear of bacterial of fungal growth due to moisture in the foam.

The apparatus comprises at least four layer wherein Top layer and bottom layer provides polygon hollow tube, one layer is wrapped called protective layer around apparatus and another layer said Foam layer is located at the bottom of said apparatus and comes in contact with skin. Composition of resin is already filled into polygon hollow pipe of said apparatus. Before removing protective layer, flexible apparatus is wrapped around fractured body part and lock by means of locking mechanism. After peeling off external wrap composition of resin start to cure by means of applying visible light on to said apparatus.

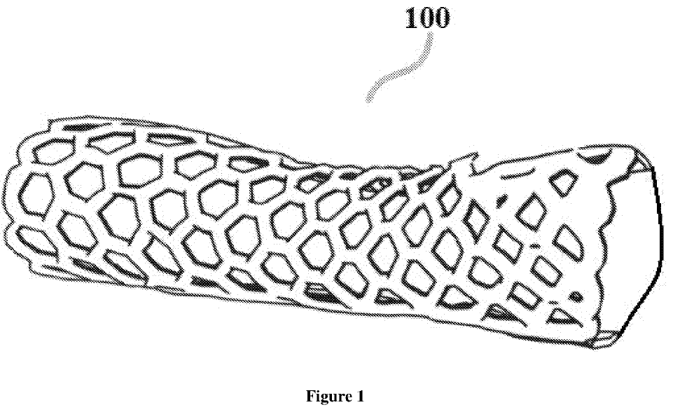

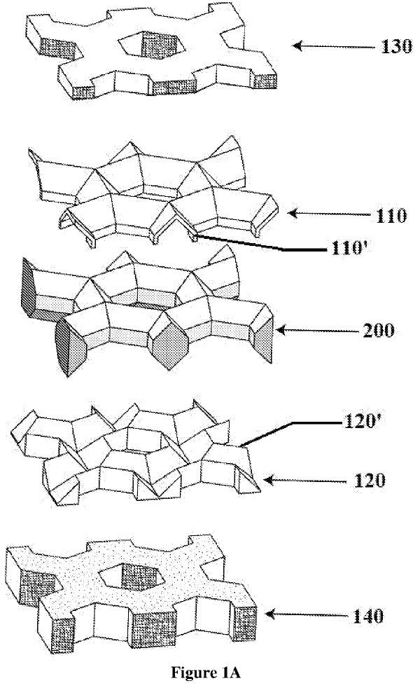

FIG. 1 of the present invention discloses a polysiloxanes orthopaedic immobilizer 100. Moreover, FIG. 1 shows one of the examples of the immobilizer of the present invention which is applied at fracture bone of hand. Further, the present invention does not limit its scope by means of providing shape of immobilizer as shown in FIG. 1 but the present invention provides an immobilizer having different shape and size; and applicable to every fracture parts of human body. Moreover, FIG. 1 of the present invention discloses an embodiment of the present invention wherein a polysiloxanes orthopaedic immobilizer 100 comprises top layer 110 and bottom layer 120 which make a hollow polygon shape tube structure consisting of hexagon, octagon, pentagon, tringle, circle, etc. further, said orthopaedic immobilizer 100 consisting removable wrapper 130 attached with the top layer 110 which can be removed to start curing process which makes rigid to the orthopaedic immobilizer 100 of the present invention. Said orthopaedic immobilizer 100 consisting of Foam layer 140 which is attached at the bottom of bottom layer 120 and protects skin against heat generating during curing process.

FIG. 1A of the present invention discloses an exploded view of the polysiloxanes orthopaedic immobilizer 100 wherein top layer 110 and bottom layer 120 configure hollow polygon shape tube preferably hexagonal hollow tube 10. The present invention does not limit its scope by providing hexagonal hollow tube configured by top layer 110 and bottom layer 120 but in the present invention top layer 110 and bottom layer 120 may configure octagon, pentagon, tringle, circle, etc shape of hollow tube 10. Further, FIG. 1A discloses filled resin volume 200 which generated by means of top layer 110 and bottom layer 120. Moreover, removable layer 130 attached with the top layer 110 which can be removed to start curing process which makes rigid to the orthopaedic immobilizer 100 of the present invention. Said orthopaedic immobilizer 100 consisting of Foam layer 140 which is attached at the bottom of bottom layer 120 and protects skin against heat generating during curing process. The word “ribs” define in the present invention as a structural member of polygon shape which help to make a polygon curve.

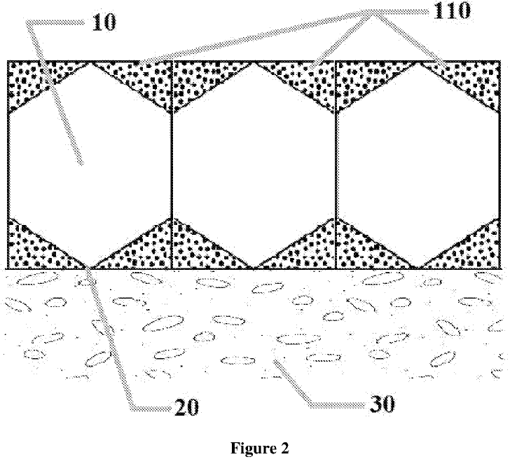

FIG. 2 of the present invention illustrate an embodiment of the present invention wherein; FIG. 2 discloses a cross-sectional view of the orthopaedic immobilizer 100. In FIG. 2, said immobilizer 100 consisting of hexagonal shaped hollow tube 10 but the present invention does not limit its scope by providing only hexagonal shaped hollow tube 10 but it can be octagon, pentagon, tringle, circle, etc. Moreover, FIG. 2 of the present invention illustrate an embodiment of the present invention wherein the most preferable shape of immobilizer 100 of the present invention because the hexagonal shaped hollow tube 10 provides point contact 20 with the skin of patient 30 as the rate of heat transfer from immobilizer 100 to the skin of patient 30 is negligible.

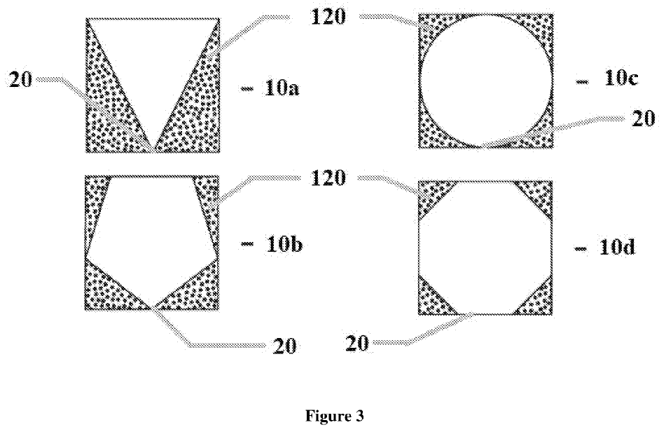

FIG. 3 of the present invention discloses an embodiment of the present invention wherein, immobilizer 100 can be shaped of triangle 10a, pentagon 10b, circle 10c and octagon 10d. But the hollow tube 10 structure can be any polygon shape as mentioned above.

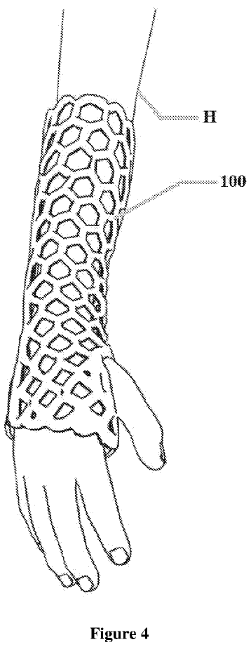

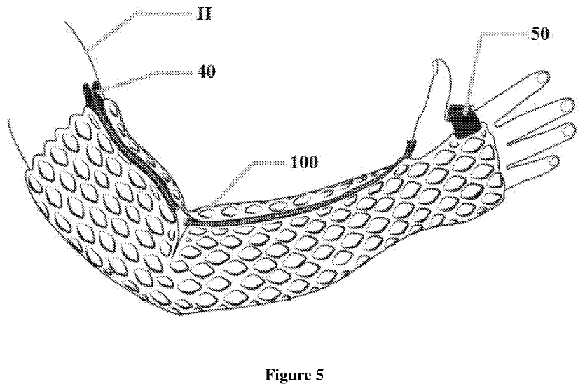

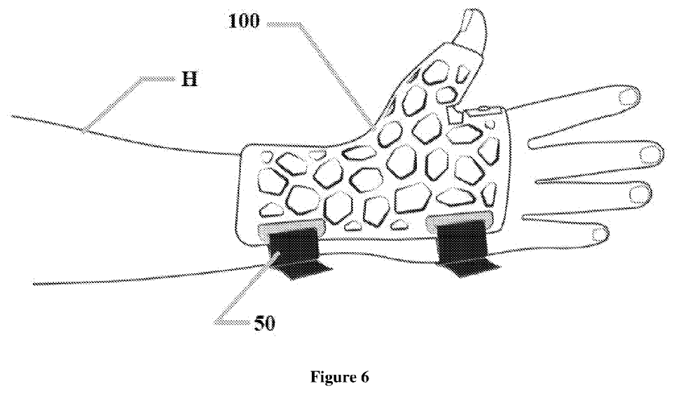

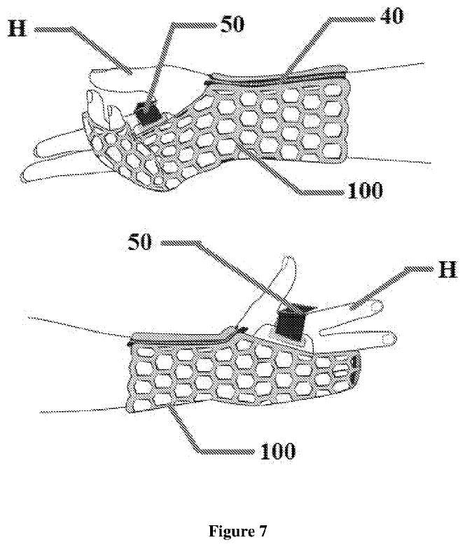

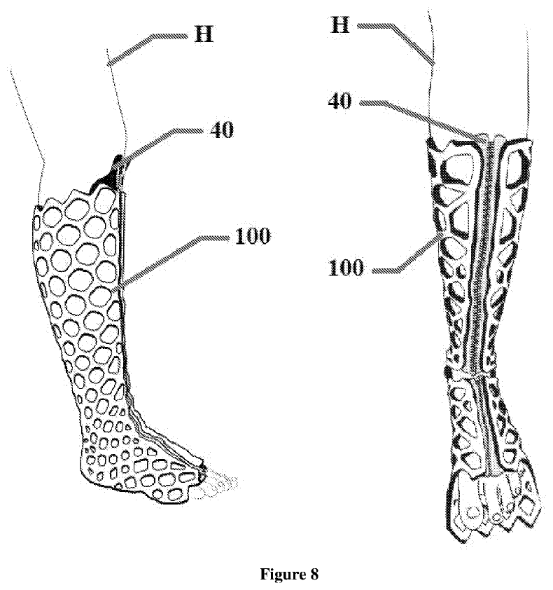

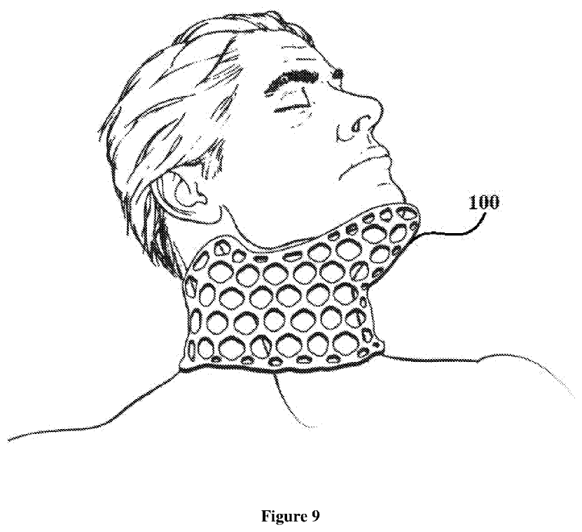

FIG. 4 of the present invention discloses an embodiment of the present invention wherein said orthopaedic immobilizer 100 is applied to the fracture limb of hand like ulna bone, radius bone or any other bone of hand H. After applied said immobilizer 100, removable wrapper 130 will be removed from the top layer 110 so that visible light can go through the bottom layer 120 of the immobilizer 100 which start curing process. After compliance of curing process, the said orthopaedic immobilizer 100 will be rigid. FIG. 5 of the present invention discloses an immobilizer 100 which is applied at fracture elbow joint of hand H of human body. One of the embodiments of the present invention is to provide an immobilizer 100 having locking mechanism 40 and fastening mechanism 50 to hold the said orthopaedic immobilizer 100 to the human body. FIG. 6 of the present invention discloses a immobilizer 100 applied at the fractured carpus by means of fastening mechanism 50. FIG. 7 of the present invention discloses an orthopaedic immobilizer 100 which is applied at the fractured metacarpals and phalanges bones. FIG. 8 of the present invention discloses an orthopaedic immobilizer which is applied at the fractured femur, fibula and tibia bones. FIG. 9 of the present invention discloses an orthopaedic immobilizer which is applied at the cervical bone of the human.

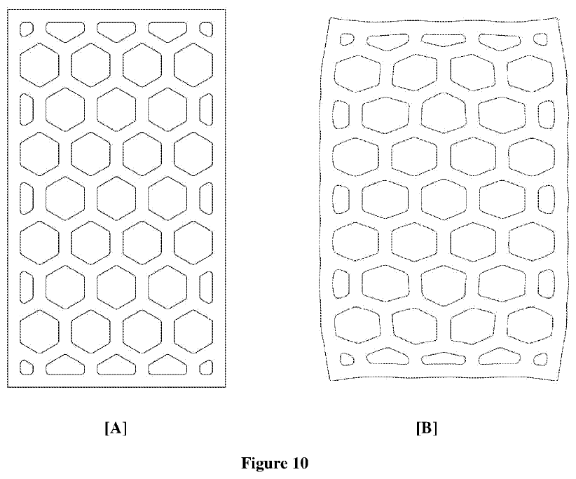

FIG. 10 of the present invention discloses an embodiment wherein an immobilizer having polygon shape hollow tubes which provide more stretch ability, stable structure and more deformation to the immobilizer having most preferably shape is hexagon. FIG. 10 [A] discloses a hexagonal shape immobilizer which is its normal state. When said hexagonal shape immobilizer has been stretched then said immobilizer looks like FIG. 10 [B] which is clearly discloses that hexagon shape provides more stretch ability, stable structure as no minor changes cause to the shape of hexagon after stretching and provide more deformation then other shape. The present invention also discloses comparison between hexagon shape and other shape i.e. circular, square, triangle, etc.

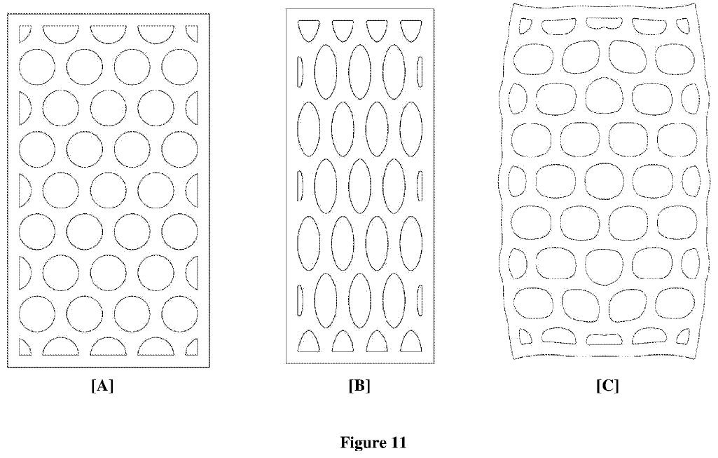

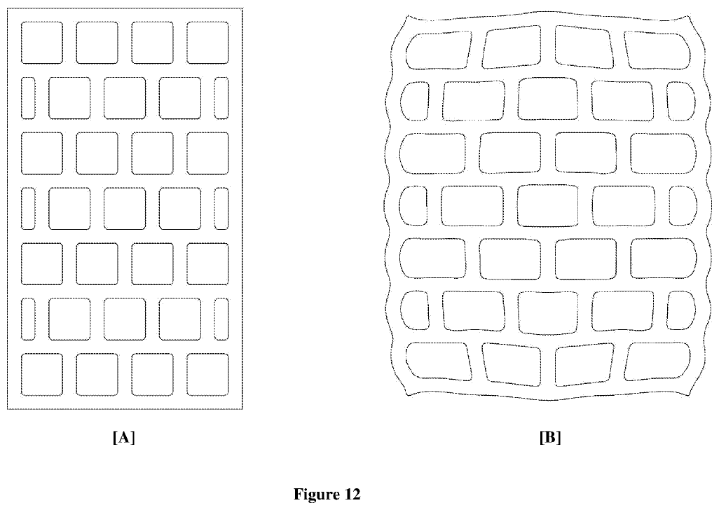

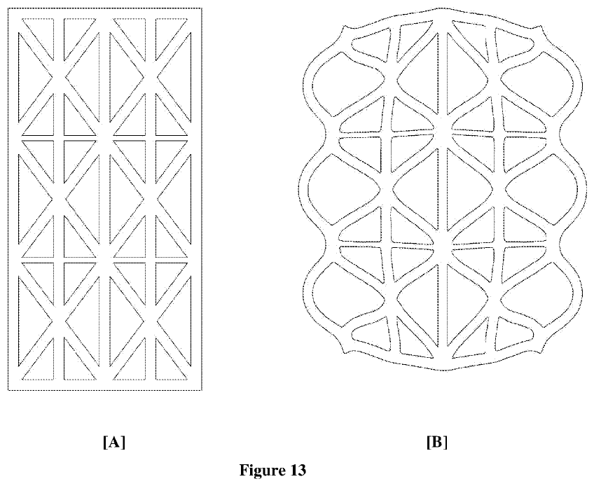

FIG. 11 discloses a circular shape immobilizer in [A]; but if tension force or compression force is applied to the said circular shape immobilizer then it will deform as shown in Figure [B] & [C] which may cause failure to the immobilizer. FIG. 12 of the present invention discloses square shape immobilizer into [A] which will stretch and looks like [B] which cause failure to the structure. FIG. 13 of the present invention discloses triangular shape immobilizer as shown in [A] which turn into the shape as shown in [B] after applying tension force. It is clearly, see that if one can stretch an immobilizer having shape of except hexagon; it causes structure failure hence most preferable shape in hexagon.

We claim:

1. A polysiloxanes orthopaedic immobilizer characterised by top layer 110 and bottom layer 120 configure polygon hollow tube 10;
Wherein,
a. polygon hollow tube 10 is pre-filled with composition of resin;
b. composition of resin is cured with exposure of visible light having wave length of 400-470 nm after peeling off protective layer 130;
c. protective layer 130 is wrapped around the top layer 120 prevents composition of resin to be cured;
d. top layer 110 comprises ribs 110′ connects with ribs 120′ of bottom layer 120;
e. bottom layer 120 comprises ribs 120′ connects with ribs 110′ of top layer 110;
f. foam layer 140 is attached at the base of bottom layer 120 and protects skin against heat generating during curing process;
wherein, polygon hollow tube 10 provides point contact 20 between skin 30 and immobilizer 100.

2. The polysiloxanes orthopaedic immobilizer as claimed in claim 1, wherein curing process starts after removing protective layer provided on outer surface of said immobilizer.

3. The polysiloxanes orthopaedic immobilizer as claimed in claim 1, wherein foam layer protects skin against heat generation at the time of polymerisation process of composition of resin.

4. The polysiloxanes orthopaedic immobilizer as claimed in claim 1, wherein hollow polygon shape tube structure consisting of hexagon, octagon, pentagon, tringle, circle, etc.

5. The polysiloxanes orthopaedic immobilizer as claimed in claim 1, having x-ray permeable tendency.

6. The polysiloxanes orthopaedic Immobilizer as claimed in claim 1, wherein polygon hollow tube 10 provides maximum light distribution tendency, viscous fluid distribution tendency and prevents direct contact of skin and immobilizer.

7. The polysiloxanes orthopaedic immobilizer as claimed in claim 1, wherein polygon hollow tube consisting of cavity wherein composition of resin is pre-filled before applying to fractured limb.

8. The polysiloxanes orthopaedic immobilizer as claimed in claim 1, wherein immobilizer made up from polysiloxanes having tendency of absorption, reflection and allows visible light to reach at the base of the said immobilizer.

9. The polysiloxanes orthopaedic Immobilizer as claimed in claim 1, Wherein immobilizer comprises padding layer made up of closed cell synthetic elastomer which gives insulation from exothermic heat produced during curing process as well as cushion to skin against hard shell formed after curing.

10. The polysiloxanes orthopaedic Immobilizer as claimed in claim 9, Wherein Closed cell foam is non-allergic and non-sensitize suitable for long contact with skin.

11. The polysiloxanes orthopaedic Immobilizer as claimed in claim 9, Wherein Closed cell foam does not absorb water so that it allows user to wash immobilizer thoroughly without fear of bacterial of fungal growth due to moisture in the foam.

12. The polysiloxanes orthopaedic immobilizer as claimed in claim 1, wherein polygon shape of tube prevents tubes from collapsing and improve structural strength of said immobilizer during wrapping around body part immobilizer.

13. The polysiloxanes orthopaedic immobilizer as claimed in claim 1, wherein immobilizer structure arranges in polygon matrix form.

14. The polysiloxanes orthopaedic immobilizer as claimed in claim 1, wherein top layer of a immobilizer and bottom layer of a immobilizer is hydrophobic.

15. The polysiloxanes orthopaedic immobilizer as claimed in claim 1, wherein different colours of dies are mixed with polysiloxanes and composition of resin for achieving aesthetic appearance of immobilizer.

16. The polysiloxanes orthopaedic immobilizer as claimed in claim 1, wherein an apparatus comprises locking mechanism which fastens said apparatus at the time of wrapping around body parts.

17. The polysiloxanes orthopaedic immobilizer as claimed in claim 1 is pre-fabricated and stretchable due to its elasticity and because of elasticity said Immobilizer can fit on to variable size difference and variable contour of body part

18. The polysiloxanes orthopaedic immobilizer as claimed in claim 1, wherein an apparatus having polygon matrix shape which is admitting air to the skin and allowing sweat to evaporate.

19. The polysiloxanes orthopaedic immobilizer as claimed in claim 1, wherein a method for forming a matrix orthopaedic immobilizer for immobilizing a body of a patient to allow healing.

20. The polysiloxanes orthopaedic immobilizer as claimed in claim 1, wherein an immobilizer having different shape and size and applicable to every fractured parts of human body.
